SEQUENCE LISTING (6 sequences)

SEQ ID NO: 1 - DNA, 21 bp, Artificial Sequence
  Forward PCR Primer
gactttgaccggaacgtgccc

SEQ ID NO: 2 - DNA, 21 bp, Artificial Sequence
  Reverse PCR Primer
catcatgccgatgtccacaca

SEQ ID NO: 3 - DNA, 23 bp, Artificial Sequence
  Forward PCR Primer
cgggtgtaccatttacaactcgg

SEQ ID NO: 4 - DNA, 23 bp, Artificial Sequence
  Reverse PCR Primer
ctcaacaggctcattgtctgtgg

SEQ ID NO: 5 - DNA, 20 bp, Artificial Sequence
  Forward PCR Primer
accacagtccatgccatcac

SEQ ID NO: 6 - DNA, 20 bp, Artificial Sequence
  Reverse PCR Primer
tccaccaccctgttgctgta

(19) United States
(12) Patent Application Publication
(10) Pub. No.: US 2007/0134286 A1
(43) Pub. Date: Jun. 14, 2007

(54) Methods for reducing intimal hyperplasia, smooth muscle cell proliferation and restenosis in mammals

(76) Inventor: Jinshyun R. Wu-Wong, Libertyville, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/292,050

(22) Filed: Dec. 1, 2005

Related U.S. Application Data
(60) Provisional application No. 60/632,555, filed on Dec. 2, 2004.

(51) Int. Cl. A61K 31/59

(52) U.S. Cl. 424/423; 514/167

(57) ABSTRACT

The present invention relates to methods for reducing small muscle cell proliferation, restenosis or intimal hyperplasia in mammals.

The subject application claims priority to U.S. provisional patent application No. 60/632,555, filed on Dec. 2, 2004, which is hereby incorporated in its entirety by reference.

### FIELD OF THE INVENTION

The present invention relates to methods for reducing smooth muscle cell proliferation or intimal hyperplasia in mammals, particularly humans, by the administration of at least one compound that activates a vitamin D receptor (hereinafter “VDR”). Compounds that activate a VDR and can be used in the methods described herein include, but are not limited to, vitamin D compounds.

### BACKGROUND OF THE INVENTION

Under normal conditions, the human body acquires vitamin D₃ via diet or by exposure to sun light. Typically, vitamin D₃ is not active and is modified by 25-hydroxylase in the liver and by 25-hydroxyvitamin D 1α-hydroxylase in the kidney to form the active metabolite, 1α,25-dihydroxyvitamin D₃, which is then metabolized by 25-hydroxyvitamin D-24-hydroxylase (hereinafter “24-OHase”) (Brown A J, Dusso A S & Slatopolsky E, “Vitamin D analogues for secondary hyperparathyroidism,” Nephrol Dial Transplant, 17:10-19 (2002); Wu-Wong J R, Tian J & Goltzman D, “Vitamin D analogs as therapeutic agents: a clinical study update,” Curr Opin Investig Drugs, 5:320-326 (2004)). The binding of 1α,25-dihydroxyvitamin D₂ or D₃ or their analogs to VDR, a nuclear receptor, activates VDR to interact with retinoid X receptor (hereinafter “RXR”) to form the VDR/RXR/cofactor complex, which binds to vitamin D response elements in the promoter region of target genes to regulate gene transcription (Carlberg C, Quack M, Herdick M, Bury Y, Polly P & Toell A, “Central role of VDR conformations for understanding selective actions of vitamin D(3) analogues,” Steroids, 66:213-221 (2001)). The VDR has been found in more than 30 tissues including intestine, bone, kidney, parathyroid gland, pancreatic β-cells, monocytes, keratinocytes, and many cancer cells, suggesting that the vitamin D endocrine system may also be involved in regulating the immune systems, cellular growth, differentiation and apoptosis.

It is well documented that 1α,25-dihydroxyvitamin D₃ (also known as 1α,25(OH)₂D₃ or calcitriol, the endogenous VDR activator) regulates the homeostasis of calcium and phosphorus. 1α,25(OH)₂D₃ is also involved in regulating the deposition of calcium and phosphorus into the bone. The kidney is the major site where 25-hydroxyvitamin D-1α-hydroxylase resides and activates vitamin D. Vitamin D deficiency results in defective intestinal absorption of calcium and phosphate.

Renal diseases, even in the early stages, often result in reduced synthesis of 1α,25(OH)₂D₃. As the renal disease progresses, inadequate renal phosphate clearance and insufficient calcium absorption occur to maintain ionized calcium levels within an optimal range, lead to chronic over-stimulation of the parathyroid gland and PTH synthesis. In addition, 1α,25(OH)₂D₃ plays a direct role in the regulation of the synthesis and secretion of PTH (Silver J, Kilav R, Naveh-Many T, “Mechanisms of secondary hyperparathyroidism,” Am J Physiol Renal Physiol, 283:F367-76 (2002)). Hypocalcemia and hyperphosphatemia upregulate PTH gene expression by affecting the stability of PTH mRNA post-transcriptionally, while 1α,25(OH)₂D₃ downregulates PTH gene expression at the transcriptional level (Silver J, Kilav R, Naveh-Many T, “Mechanisms of secondary hyperparathyroidism,” Am J Physiol Renal Physiol, 283:F367-76 (2002)). Consequently, secondary hyperparathyroidism (hereinafter “SHPT”) is a frequent complication in chronic renal disease characterized by parathyroid hyperplasia, and enhanced synthesis and secretion of PTH (Slatopolsky E, Brown A, Dusso A, “Pathogenesis of secondary hyperparathyroidism,” Kidney Int, 73:S14-19 (1999)). In addition, chronic renal disease patients often experience renal osteodystrophy characterized by high, low or mixed turnover bone diseases. PTH is considered a uremic toxin that exerts its effects on multiple organs, and controlling the PTH level is a crucial step in the management of chronic renal disease. VDR activators such as 1α,25-dihydroxyvitamin D₃, and 19-nor-1α, 25-dihydroxyvitamin D₂ (an analog of 1α,25-dihydroxyvitamin D₃ with reduced calcemic side effect) are now routinely used to manage SHPT. The VDR activators control parathyroid function via both direct and indirect effects. VDR activators directly down-regulate PTH transcription and synthesis and also directly inhibit parathyroid cell proliferation. These agents improve skeletal resistance to the calcemic action of PTH, and improves calcium homeostasis, which then indirectly control PTH function. It is important to emphasize that normal kidney function is required for maintaining the 1α,25(OH)₂D₃ level. CKD patients need VDR activators to treat SHPT. Vitamin D supplementation, no matter how high the level is, will not benefit CKD patients. Furthermore, it is important to consider normal liver function when treating CKD patients with pro-drugs such as 1α(OH)D₂ or D₃, which requires 25-hydroxylase in the liver to become activated into 1α,25(OH)₂D₂ or D₃.

Patients suffering from chronic kidney disease (abbreviated as “CKD”) slowly lose kidney function over a period of time. CKD is currently defined as kidney damage, confirmed by a kidney biopsy or characterized by markers of kidney damage, or a glomerular filtration rate (abbreviated as “GFR”)<60 mL/min/1.73 m² for three months. Kidney damage is defined as pathological abnormalities or makers of damage, including abnormalities in blood or urine tests or imaging studies. Markers of kidney damage include proteinuria, abnormalities on the urine dipstick or sediment examination, or abnormalities on imaging studies of the kidneys. GFR can be estimated from prediction equations based on serum creatinine and other variables, including age, sex, race, and body size.

Among individuals with CKD, the stage of the disease (see below Table 1 which is taken from the National Kidney Foundation Kidney Disease Quality Initiative (K/DOQI), “Clinical Practice Guidelines for Bone Metabolism in Chronic Kidney Disease,” American Journal of Kidney Diseases, 42(4), Supp. 3, S1-S201 (October 2003)) is based on the level of GFR, irrespective of the cause of kidney disease.

TABLE 1

| Stage | | GFR (mL/min/1.73 m²) |
|---|---|---|
| 1 | Kidney damage with normal or | ≧90 |
| | increased GFR | |
| 2 | Kidney damage with mild decrease | 60-89 |
| | in GFR | |
| 3 | Moderate decrease in GFR | 30-59 |
| 4 | Severe decrease in GFR | 15-29 |
| 5 | Kidney Failure (End-Stage Renal | <15 (or dialysis) |
| | Disease) | |

CKD patients become unable to make metabolically active vitamin D and become inefficient at excreting phosphate. As a result, their levels of metabolically active vitamin D drop, circulating blood calcium levels drop, and circulating blood phosphate levels increase. In an attempt to compensate for the change in circulating blood calcium and phosphate levels, the parathyroid gland secretes PTH to normalize the calcium and phosphate levels. Eventually, the secretion of PTH becomes excessive. This excessive secretion of PTH is referred to as secondary hyperparathyroidism (abbreviated as “SHPT”). Additionally, patients with PTH levels higher than those in the recommended range are at greater risk for bone disorders. The National Kidney Foundation Kidney Disease Outcomes Quality Initiative (K/DOQI) guideline (“Clinical Practice Guidelines for Bone Metabolism in Chronic Kidney Disease,” American Journal of Kidney Diseases, 42(4), Supp. 3, S1-S201 (October 2003)) recommends treatment when PTH levels are greater than 70 pg/mL to prevent or ameliorate bone disease.

By the time these patients reach the end stage of the disease (known as “end stage renal disease” or “ESRD”), their kidneys function less than 10% of the baseline and are no longer able to function at the level necessary for day-to-day life. At this point, these patients need to undergo dialysis treatment or receive a kidney transplant.

Before hemodialysis is started in a patient suffering from CKD, a vascular access site must be prepared. A vascular access site is a site on the body where blood is removed and returned during dialysis. Three types of vascular accesses are commonly used in CKD patients. These are an arteriovenous (AV) fistula, an AV graft and a venous catheter. Each of these vascular accesses can cause complications that require further treatment or surgery. The most common complications are access infection and failure. Access failure is primarily due to stenosis which is the result of intimal hyperplasia (which mainly involves smooth muscle cell proliferation), inflammation and thrombosis. Access failure can be resolved via angioplasty, which is a procedure that is used to widen a small segment of a blood vessel that has narrowed. Alternatively, another option is to perform surgery and replace the narrow segment of the blood vessel.

Stenosis not only affects CKD patients undergoing hemodialysis, but any patient suffering from a vascular disease that obstructs or narrows a blood vessel. For example, injury to a blood vessel, such as a coronary artery, due to cholesterol, intimal hyperplasia, inflammation and thrombosis, can, over time, result in the narrowing of a blood vessel. Balloon angioplasty can be used to widen or open an obstructed or narrowed blood vessel.

Stenosis can also occur after a bypass graft operation, such as after a coronary artery bypass graft. This type of surgery is done to reroute, or “bypass” blood around clogged blood vessels, particularly, arteries. In this situation, stenosis can occur in the transplanted blood vessel segments. Like other stenosed blood vessels, angioplasty or atherectomy may be need to “widen” or “reopen” the blood vessels.

A typical angioplasty procedure involves the following steps. First, a surgeon threads a narrow catheter (a tube) containing a fiber optic camera directly to the blocked blood vessel. Next, the surgeon opens the blocked vessel using a balloon angioplasty, wherein the surgeon passes a tiny deflated balloon through the catheter to the blood vessel. The balloon is then inflated to compress the plaque against the wall of the blood vessel, flattening it out so that blood can once again flow through the vessel freely. In order to keep the artery open afterwards, the surgeon commonly employs a device called a “stent”, which is an expandable metal or polymer mesh tube that is implanted during angioplasty at the site of the blockage. Once in place, the stent pushes against the wall of the blood vessel to keep it open.

Studies report high survival rates with the use of stents, including their use with multiple blood vessels. However, in many cases, stented blood vessels eventually become occluded due to restenosis (renarrowing). Restenosis is the blood vessel's natural response to injury following stenting or balloon angioplasty. Approximately one-third of patients who undergo angioplasty have restenosis of the widened segment within about six months of the procedure. Restenoses arteries may have to undergo another angioplasty. To reduce the incidents of restenosis, sometimes stents are coated or embedded with drugs.

CKD patients encounter a much higher risk of cardiovascular disease. Data from the Unites States Dialysis Registry show that the risk of cardiovascular death in the young (25-34 years) dialysis patient group is 500 times higher than that in the age-matched general population. Even in the age segment 45-55 years, it is still 60 times higher than the normal annual mortality. In fact, the absolute risk of a CKD patient to die from cardiovascular disease is higher than that of a patient who has had a previous myocardial infarction. From a study correlating CKD, mortality and treatment strategies among patients with clinically significant coronary artery disease, it was shown that each 10 ml/mi decrement in GFR was associated with a >10% increase in mortality in Stage 2 CKD. In Stages 3 and 4, the survival probability after 5 years is reduced to <50% when compared to normal individuals. The high prevalence of vascular disease in CKD has been well documented. In a study to assess the morphology of coronary arteries in patients with end-stage renal failure and compared them with coronary arteries of matched non-uremic control patients, it was found that the coronary arteries in patients with end-stage renal failure showed significantly more calcified plaques. Medial thickness of coronary arteries was significantly higher in uremic patients. Lumen area was significantly lower in uremic patients.

Thereupon, there is a need in the art for methods for reducing smooth muscle cell proliferation in a mammal, particularly in a mammal suffering from vascular disease, such as coronary artery disease. Additionally, there is a further need in the art for reducing intimal hyperplasia in mammals suffering from chronic kidney disease and/or in mammals that have undergone coronary artery bypass surgery. Finally, there is also a need in the art for reducing restenosis in a mammal that has undergone angioplasty.

### SUMMARY OF THE INVENTION

In one embodiment, the present invention relates to a method of reducing smooth muscle cell proliferation in a mammal suffering from a vascular disease, wherein said vascular disease causes a narrowing or obstruction of a blood vessel. The method comprises the step of administering to said mammal a therapeutically effective amount of a compound that activates a vitamin D receptor (VDR). These compounds can be administered orally or intravenously or as part of a coating on a stent or as part of a coating on a graft or implant. Compounds that activate a vitamin D receptor include vitamin D compounds, such as, but not limited to, vitamin D metabolites or analogs (such as 1,25-dihydroxyvitamin D₃ or 19-nor-1α,25-dihydroxyvitamin D₂). If a vitamin D compound is to be administered, it can be administered in the amount of from about 0.04 to about 0.24 mcg/kg, intravenously, three times per week.

In another embodiment, the present invention relates to a method of reducing intimal hyperplasia in a mammal suffering from chronic kidney disease. The method comprises the step of administering to said mammal a therapeutically effective amount of a compound that activates a VDR. These compounds can be administered orally or intravenously or as part of a coating on a stent or as part of a coating on a graft or implant. Compounds that activate a vitamin D receptor include vitamin D compounds, such as, but not limited to, vitamin D metabolites or analogs (such as 1,25-dihydroxyvitamin D₃ or 19-nor-1α,25-dihydroxyvitamin D₂). If a vitamin D compound is to be administered, it can be administered in the amount of from about 0.04 to about 0.24 mcg/kg, intravenously, three times per week.

In yet a further embodiment, the present invention relates to a method of reducing restenosis in a mammal that has undergone angioplasty. The method comprises the step of administering to said mammal a therapeutically effective amount of a compound that activates a VDR. These compounds can be administered orally or intravenously or as part of a coating on a stent or as part of a coating on a graft or implant. Compounds that activate a vitamin D receptor include vitamin D compounds, such as, but not limited to, vitamin D metabolites or analogs (such as 1,25-dihydroxyvitamin D₃ or 19-nor-1α,25-dihydroxyvitamin D₂). If a vitamin D compound is to be administered, it can be administered in the amount of from about 0.04 to about 0.24 mcg/kg, intravenously, three times per week.

In still yet a further embodiment, the present invention relates to a method of reducing intimal hyperplasia in a mammal that has undergone coronary artery bypass surgery. The method comprises the step of administering to said mammal a therapeutically effective amount of a compound that activates a VDR. These compounds can be administered orally or intravenously or as part of a coating on a stent or as part of a coating on a graft or implant. Compounds that activate a vitamin D receptor include vitamin D compounds, such as, but not limited to, vitamin D metabolites or analogs (such as 1,25-dihydroxyvitamin D₃ or 19-nor-1α,25-dihydroxyvitamin D₂). If a vitamin D compound is to be administered, it can be administered in the amount of from about 0.04 to about 0.24 mcg/kg, intravenously, three times per week.

In yet a further embodiment, the present invention relates to a method of reducing intimal hyperplasia in a mammal suffering from chronic kidney disease that has a need for hemodialysis which requires implementation of a vascular access device. The method comprises the step of administering to said mammal a therapeutically effective amount of a compound that activates a VDR. These compounds can be administered orally or intravenously or as part of a coating on a stent or as part of a coating on a graft or implant. Compounds that activate a vitamin D receptor include vitamin D compounds, such as, but not limited to, vitamin D metabolites or analogs (such as 1,25-dihydroxyvitamin D₃ or 19-nor-1α,25-dihydroxyvitamin D₂). If a vitamin D compound is to be administered, it can be administered in the amount of from about 0.04 to about 0.24 mcg/kg, intravenously, three times per week.

### BRIEF DESCRIPTION OF THE FIGURES

FIG. 1 shows the characterization of VDR in human coronary artery smooth muscle cells by ³H-calcitriol binding to cell extract prepared from these cells.

FIG. 2 shows the results from confocal microscopy. More specifically, VDR activator (such as paricalcitol and calcitriol) binding results in the translocation of VDR into the cellular nucleus, although not all cells respond (VDR (red)/RXR (green)).

FIGS. 3 and 4 show that VDR activators, such as the vitamin D compounds, paricalcitol and calicitrol, despite having different characteristics, can be used to inhibit reduce smooth muscle cell proliferation in mammals. These figures show that paricalcitol is slightly more potent than calcitriol in inhibiting DNA synthesis in these cells.

FIG. 5 shows the effect of VDR activators, such as paricalcitol and calicitriol, on inducing the synthesis of 24-OHase mRNA in these cells. The potency is in the order of calcitriol>paricalcitol.

FIG. 6 shows in the top panel a Western Blot. Plasminogen activator inhibitor type-1 (PAI-1) is one of the risk markers associated with coronary heart disease and is enhanced in atherosclerotic plaque and colocalized with macrophages. The bottom panel in FIG. 4 is a bar graph that shows that paricalcitol is as potent as calcitriol in inhibiting PAI-1 expression.

FIG. 7 illustrates the effects of paricalcitol on IGF1, WT1, and TGFβ3 mRNA expression. Smooth muscle cells were harvested, RNA isolated and the mRNA levels of target genes analyzed by real-time RT-PCR. GADPH was used for normalization.

FIG. 8 illustrates the effect of paricalcitol and calcitriol on cell proliferation. Cells were treated with increasing concentrations of paricalcitol or calcitriol as described in Example 2. Data were expressed as % of control (cells in growth medium without drug treatment, 100%). Each value shown is mean ± the standard deviation (n=4-8 from combining two independent experiments). Serum free medium was used as a comparison in the studies (21.4±5.3% of control). Statistical comparisons were performed by unpaired t-test. **p<0.01, ***p<0.001 compared with control.

FIG. 9 illustrates the effects of vitamin D analogs in the blood vessel. VDR activation by vitamin D analogs results in regulation of genes involved in the cell cycle that leads to inhibition of proliferation and induction of differentiation. BNP (natriuretic peptide-B) is down-regulated. Regulation of genes such as TM, thrombospondin 1 and MMP by vitamin D analogs results in reduced thrombogenicity and increased fibrinolysis. The regulation of type-B endothelin receptor, oxytocin receptor, and prostaglandin-endoperoxide synthase 1 suggest that vitamin D analogs may also play roles in vessel relaxation and endothelial regeneration.

### DETAILED DESCRIPTION OF THE INVENTION

Definitions

As used herein, the term “intimal hyperplasia” refers to a proliferative response of smooth muscle cells (SMCs) to a vascular injury which results in intimal and medial thickness of arteries and a narrowing of the lumen area. The injury could be from a vascular intervention such as, angioplasty, endarterectomy, the placement of vascular access in a CKD patient, etc. The injury could also occur due to diseases such as hypertension, diabetes or dyslipidemia. The hyperplastic growth gradually encroaching into the lumen of the blood vessel is the leading cause of stenosis (or restenosis). Hyperplasia occurs gradually over a period of days to several weeks following the arterial intervention, as distinguished from a thrombus, such as may occur in the circulating blood immediately at the time of intervention.

More specifically, intimal hyperplasia is caused by a cascade of events in response to vascular damage. As part of the inflammatory and reparative response to vascular damage, such as that resulting from vascular surgeries, inflammatory cells (such as monocytes, macrophages, and activated polymorphonuclear leukocytes and lymphocytes) often form inflammatory lesions in the blood vessel wall. Lesion formation activates cells in the intimal and medial cellular layers of the blood vessel or heart. The cellular activation may include the migration of cells to the innermost cellular layers, known as the intima. Such migrations pose a problem for the long-term success of vascular grafts because endothelial cells release smooth muscle cell growth factors (e.g., platelet-derived growth factor, interleukin-1, tumor necrosis factor, transforming growth factor-beta, and basic fibroblast growth factor), that cause these newly-migrated smooth muscle cells to proliferate. Additionally, thrombin has been demonstrated to promote smooth muscle cell proliferation both by acting as a growth factor itself and by enhancing the release of several other growth factors produced by platelets and endothelial cells (Wu et al., Annu. Rev. Med. 47:315-31 (1996)). Smooth muscle cell proliferation causes irregular and uncontrolled growth of the intima into the lumen of the blood vessel or heart, which constricts and often closes the vascular passage. Often, irregular calcium deposits in the media or lipid deposits in the intima accompany smooth muscle cell growths, such lipid deposits normally existing in the form of cholesterol and cholesteryl esters that are accumulated within macrophages, T lymphocytes, and smooth muscle cells. These calcium and lipid deposits cause arteriosclerotic hardening of the arteries and veins and eventual vascular failure. These arteriosclerotic lesions caused by vascular grafting can also be removed by additional reconstructive vascular surgery, but the failure rate of this approach due to restenosis has been observed to be between thirty and fifty percent.

As used herein, the phrase “a compound that activates a vitamin D receptor” and the term “VDR activator” are used interchangeably. As used herein, the phrase “a compound that activates a vitamin D receptor” and the term “VDR activator” refer to any compound that is capable of binding to a vitamin D receptor and eliciting a functional response in the VDR signal pathway. Examples of VDR activators include, but are not limited to, vitamin D compounds, as well as any other compounds that are capable of binding to a vitamin D receptor and eliciting a functional response in the VDR signal pathway but are structurally different from vitamin D compounds.

As used herein, the term “vitamin D compound” encompasses compounds which control one or more of the various vitamin D-responsive processes in mammals, i.e., intestinal calcium absorption, bone mobilization, bone mineralization and cell differentiation. Thus, the vitamin D compounds encompassed by this invention include cholecalciferol and ergocalciferol and their metabolites, as well as the synthetic cholecalciferol and ergocalciferol analogs which express calcemic or cell differentiation activity. Without limiting the vitamin D compounds encompassed by the present invention, these synthetic cholecalciferol and ergocalciferol analogs comprise such categories of compounds as the 5,6 trans-cholecalciferols and 5,6-trans-ergocalciferols, the fluorinated cholecalciferols, the side chain homologated cholecalciferols and side chain homologated 22 cholecalciferols, the side chain-truncated cholecalciferols, the 19-nor cholecalciferols and ergocalciferols and the 10,19-dihydovitamin D compounds.

Some specific examples of such compounds include, but are not limited to, vitamin D metabolites or analogs such as vitamin D₃, vitamin D₂, 1-hydroxyvitamin D₃, 1-hydroxyvitamin D₂, 1,25-dihydroxyvitamin D₃, 1,25-dihydroxyvitamin D₂, 25-hydroxyvitamin D₃, 25-hydroxyvitamin D₂, 24,24-difluoro-25-hydroxyvitamin D₃, 24,24-difluoro-25-dihydroxyvitamin D₃, 2-fluoro-25-hydroxyvitamin D₃, 2-fluoro-1,25-dihydroxyvitamin D₃, 26, 26, 26, 27,27,27-hexafluoro-25-hydroxyvitamin D₃, 26, 26, 26, 27,27,27-hexafluoro-1,25-hydroxyvitamin D₃, 24-25-dihydroxyvitamin D₃, 14,25-trihydroxyvitamin D₃, 5,26-dihydroxyvitamin D₃, 15,26-trihydroxyvitamin D₃, 3,25-dihydroxyvitmain D₃, 23,25,26-trihydroxyvitamin D₃ and the corresponding 1-hydroxylated forms, 25-hydroxyvitamin D₃, -26,23-lacton and its 1-hydroxylated derivative, the side chain, nor, dinor, trinor and tetranor-analogs of 25-hydroxyvitamin D₃, and of, -dihydroxyvitamin D₃, 1-hydroxypregnacalciferol, and its homo and dihomo derivatives, 1,25-dihydroxy-24-20i-vitamin D₂, 24-homo-1,25,dihydroxyvitmain D₃, 24-dihomo-1,25-dihydroxyvitmain D₃, 24-trihomo-1,25-dihydroxyvitamin D₃, and the corresponding 26- or 26,27-homo, dihomo or trihomo analogs of 1,25-dihydroxyvitmain D₃, as well as the corresponding 19-nor compounds of those listed above.

As used herein, the phrase “vascular disease that causes a narrowing or obstruction of a blood vessel” refers to any vascular disease that has the effect of narrowing or obstructing a blood vessel. This phrase includes blood vessel stenosis, blood vessel restenosis, atherosclerosis, as well as obstruction caused by intimal hyperplasia.

The present invention relates to the discovery that compounds that activate a vitamin D receptor (VDR), such as, but not limited to one or more vitamin D compounds, can be used to reduce smooth muscle cell proliferation or intimal hyperplasia in mammals, preferably humans, who are in need of treatment (i.e., a human patient).

More specifically, in one embodiment, the present invention relates to a method of reducing smooth muscle cell proliferation in a mammal that is suffering from a vascular disease. The vascular disease can be any vascular disease (i.e, peripheral arterial disease, abdominal aortic aneurysm, carotid disease, venous disease, etc.) that may be causing a narrowing or obstruction (whether a partial obstruction or total obstruction) of a blood vessel (i.e., an artery). For example, the narrowing or obstruction of a blood vessel might be the result of a lesion, stenosis, restenosis, intimal hyperplasia, etc. The method involves administering to a mammal suffering from such vascular disease and in need of treatment, an effective amount of a compound that activates a VDR.

The compound can be administered in a variety of different ways. For example, the compound can be administered orally or intravenously. Alternatively, the compound can be released from a surgical or medical device or implant, such as stents (i.e., a drug coated stent), grafts (i.e, such as a vascular access graft (i.e., arteriovenous (AV) fistula, an AV graft or a venous catheter) or a bypass graft (such as a coronary artery bypass graft (CABG)), catheters, sutures, prosthesis and the like. The device or implant can be coated, embedded or impregnated with the compound. The compound may also be formed as a film. Methods for making stents and grafts, including stents and grafts that are coated, embedded or impregnated with a compound, are well known in the art and are described in U.S. Pat. Nos. 6,652,581, 6,797,727 and 6,808,536.

The compound to be administered pursuant to the method described herein can be formulated following techniques known in the art and suitable for administration via the selected route. For example, any pharmaceutically acceptable formulation containing the compound may be used, including, but not limited tablets, solutions, powders, suspension, creams, aerosols, etc. Any pharmaceutically acceptable carriers known or anticipated in the art may be added to the formulation.

An example of a compound that can be administered is a vitamin D compound. Preferably, the vitamin D compound is paricalcitol (which is also known as 19-nor-1α,3β,25-trihydroxy-9,10-secoergosta-5(Z); 7(E),22(E)-triene, 1α, 25 dihyroxy 19 nor ergocalciferol, 19-nor-1α, 25-dihydroxyvitamin D₂ and 1,α, 25-dihydroxyl-19 nor-vitamin D₂) or calcitriol (which is also known as 1-alpha-25-dihydroxyvitamin D₃). The vitamin D compound can be formulated following techniques known in the art and suitable for administration via the selected route. For example, oral capsules are disclosed in U.S. Pat. No. 4,341,774 and formulations suitable for intravenous administration are disclosed in U.S. Pat. No. 4,308,264 and WO 96/36340. Preferably, the vitamin D compound is administered in a therapeutically effective amount of from about 0.04 to about 0.24 mcg/kg, intravenously, 3 times per week, depending upon the vitamin D compound administered.

In a second embodiment, the present invention relates to a method of reducing intimal hyperplasia in a mammal that is suffering from a CKD. The method involves administering to a mammal suffering from CKD and in need of treatment, an effective amount of a compound that activates a VDR.

The compound can be administered in a variety of different ways. For example, the compound can be administered orally or intravenously. Alternatively, the compound can be released from a surgical or medical device or implant, such as stents (i.e., a drug coated stent), grafts (i.e, such as a vascular access graft for those patients in need of hemodialysis (i.e., AV fistula, an AV graft or a venous catheter) or a bypass graft (such as a CABG), catheters, sutures, prosthesis and the like. The device or implant can be coated, embedded or impregnated with the compound. The compound may also be formed as a film. As discussed previously herein, methods for making stents and grafts, including stents and grafts that are coated, embedded or impregnated with a compound, are well known in the art.

The compound to be administered pursuant to the method described herein can be formulated following techniques known in the art and suitable for administration via the selected route. For example, any pharmaceutically acceptable formulation containing the compound as discussed in connection with the previously described method herein may be used and any pharmaceutically acceptable carriers known or anticipated in the art may be added to the formulation.

An example of a compound that can be administered is a vitamin D compound. Preferably, the vitamin D compound is paricalcitol or calcitriol. The vitamin D compound can be formulated following techniques known in the art and suitable for administration via the selected route as discussed in connection with the previously described method herein. Preferably, the vitamin D compound is administered in a therapeutically effective amount of from about 0.04 to about 0.24 mcg/kg, intravenously, 3 times per week, depending upon the vitamin D compound administered.

In a third embodiment, the present invention relates to a method of reducing restenosis in a mammal that has undergone angioplasty. The method involves administering to a mammal that has undergone angioplasty an effective amount of a compound that activates a VDR.

The compound can be administered in a variety of different ways. For example, the compound can be administered orally or intravenously. Alternatively, the compound can be released from a surgical or medical device or implant, such as stents (i.e., a drug coated stent), grafts (i.e, a bypass graft such as a CABG), catheters, sutures, prosthesis and the like. The device or implant can be coated, embedded or impregnated with the compound. The compound may also be formed as a film. As previously discussed herein, methods for making stents and grafts, including stents and grafts that are coated, embedded or impregnated with a compound, are well known in the art.

The compound to be administered pursuant to the method described herein can be formulated following techniques known in the art and suitable for administration via the selected route. For example, any pharmaceutically acceptable formulation containing the compound as discussed in connection with the previously described method herein may be used and any pharmaceutically acceptable carriers known or anticipated in the art may be added to the formulation.

An example of a compound that can be administered is a vitamin D compound. Preferably, the vitamin D compound is paricalcitol or calcitriol. The vitamin D compound can be formulated following techniques known in the art and suitable for administration via the selected route as discussed with the previous method described herein. Preferably, the vitamin D compound is administered in a therapeutically effective amount of from about 0.04 to about 0.24 mcg/kg, intravenously, 3 times per week, depending upon the vitamin D compound administered.

The following non-limiting examples are presented in order to further illustrate the invention:

### EXAMPLE 1

### Reduction of Smooth Muscle Cell Proliferation Using Vitamin D Activators

Materials and Methods

Cell culture: Primary cultured human coronary artery smooth muscle cells (hereinafter “CASMC”) were purchased from Cambrex (Walkesville, Md.) and were grown to confluence in SmGM-2 (Cambrex) containing 5.5 mM glucose, 5% FBS, 50 μg/ml gentamicin, 50 ng/ml amphotericin-B, 5 μg/ml insulin, 2 ng/ml human recombinant fibroblast growth factor (hereinafter “hFGF”), and 0.5 ng/ml human recombinant epidermal growth factor (hereinafter “hEGF”) at 37° C. in a humidified 5% CO2-95% air atmosphere. Cells were grown to >80% confluence and used within five passages.

Thvmidine incorporation: Cells were plated at 1×10⁶ cells/ml, 200 μl/well into 96-well plates (Costar 3595—Cole-Parmer Instrument Company, Vernon Hills, Ill.). One day after plating, the cells were treated with test agents for 24 hours and then labeled with 0.3 μCi/well of ³H-thymidine for another 48 hours. Each well was washed with 0.3 ml/well of PBS, incubated with 0.2 ml/well of ice-cold 10% trichloroacetic acid (hereinafter “TCA”) for 30 minutes at 4° C., and then followed by another wash of 0.2 ml/well of TCA. Samples were dissolved in MICROSCINT™ 20 before counting. Data shown are mean ± the standard deviation (n=4).

RT-PCR: Cells were harvested and RNA was isolated and analyzed for RNA levels for the genes of interest and for glyceraldehyde-3-phosphate dehydrogenase (hereinafter “GAPDH”) by semi-quantitative RT-PCR. Briefly, total RNA was first reverse transcribed to cDNA using an oligo dT as a primer (Invitrogen, Carlsbad, Calif.). The cDNA samples were then amplified by PCR at 94° C. for 30 seconds/55° C. for 30 seconds/72° C. 1 minutes for 30 cycles using specific PCR primers shown below in Table 2.

TABLE 2

| | | Product | |
|---|---|---|---|
| Name | Sequence # | size | Primers |
| Human | NM000376 | 227 bp | 5′-GACTTTGACCGGAACGTGCCC-3′ |
| VDR | | | (forward); |
| | | | 5′-CATCATGCCGATGTCCACACA-3′ |
| | | | (reverse) |
| Human | L13286 | 316 bp | 5′-CGGGTGTACCATTTACAACTCGG-3′ |
| 24- | | | (forward); |
| OHase | | | 5′-CTCAACAGGCTCATTGTCTGTGG-3′ |
| | | | (reverse) |
| GAPDH | BC014085 | 450 bp | 5′-ACCACAGTCCATGCCATCAC-3′ |
| | | | (forward); |
| | | | 5-TCCACCACCCTGTTGCTGTA-3′ |
| | | | (reverse) |

SDS-PAGE and Western Blot Analysis: Cells (1×10⁶ cells per sample) pretreated with or without test agents were solubilized in 50 μl of SDS-PAGE sample buffer (Sigma, St. Louis, Mo.), and the protein content in each sample was determined by the Pierce (Rockford, Ill.) BCA protein assay. Samples were resolved by SDS-PAGE using a 4-12% NuPAGE gel (Invitrogen, Carlsbad, Calif.), and proteins were electrophoretically transferred to polyvinylidene fluoride (hereinafter “PVDF”) membrane for Western blotting. The membrane was blotted for 1 hour at 25° C. with 5% nonfat dry milk in PBS-T and then incubated with a mouse anti-PAI-1 monoclonal antibody (1000-fold dilution, Santa Cruz Biotechnology, Santa Cruz, Calif.) in PBS-T overnight at 4° C. The membrane was washed with PBS-T and incubated with a horseradish peroxidase-labeled anti-mouse antibody for 1 hour at 25° C. The membrane was then incubated with detection reagent (SuperSignal WestPico, Pierce, Rockford, Ill.). Specific bands were visualized by exposing the paper to Kodak BioMax films.

Immunostaining for VDR/RXR and Confocal Microscopy: Cells grown in four-chamber slides were treated with test agent at 0.1 μM for different periods of time. Cells were washed with PBS for 30 seconds, fixed with a fixing solution (4% formaldehyde in PBS) for 15 minutes, washed again with PBS, and then treated with 0.2% Triton X-100 in PBS for 5 minutes. The slides were rinsed with PBS and incubated with PBS plus 1% BSA for 1 hour at room temperature. The slides were then incubated with a mouse anti-VDR monoclonal antibody and a rabbit anti-RXR polyclonal antibody (50-fold dilution, Santa Cruz Biotechnology) in PBS for >20 hours at 4° C. After incubation, slides were rinsed with PBS, blocked with 1% BSA in PBS for 15 minutes, and then incubated with secondary antibodies (Alexa Fluro goat anti-mouse 568; Alexa Fluro goat anti-rabbit 488; Molecular Probes/Invitrogen) for 1 hour at 37° C., followed by another wash with PBS. The slides were mounted and photographed with a confocal microscope linked to an image analyzer.

Vitamin D Receptor (VDR) Binding Assay: Cells were collected, washed with phosphate-buffered saline, and then resuspended in hypertonic buffer (KTEDM: 300 mM KCl, 10 mM Tris, 1 mM EDTA, 5 mM DTT and 10 mM sodium molybdate, pH 7.4) followed by homogenization using an ultrasonic cell disruptor (Branson, Niantic, Conn.). The mixture was centrifuged at 207,000×g for 30 minutes. The supernatant was incubated with was incubated with increasing concentrations of [³H]Calcitriol (0.125 nM-20 nM) in KTEDM for 19-24 hours at 4° C. Non-specific binding was determined in the presence of 10 μM unlabeled calcitriol. The separation of bound and free radiolabeled ligands was achieved by addition of hydroxylapatite (hereinafter “HAP”). Briefly, the 50% slurry of HAP was prepared by suspending, washing and resuspending 10 g of Bio-Gel HAP (Bio-Rad Laboratories, Richmond, Calif.) in 60 ml of Wash Buffer (50 mM Tris/HCl, pH 7.5, with 0.5% Triton X-100) as described previously (Wecksler W R, Norman A W. Analytical Biochemistry 1979, 92:314-323). For each assay tube, an equal volume of a 50% slurry of HAP was added. The mixture was centrifuged at 12,000g for 1 minute. The pellets were washed three times with Wash Buffer. The final pellets were resuspended in 400 μl ethanol and transferred to scintillation vials for determination of radioactivity.

Results

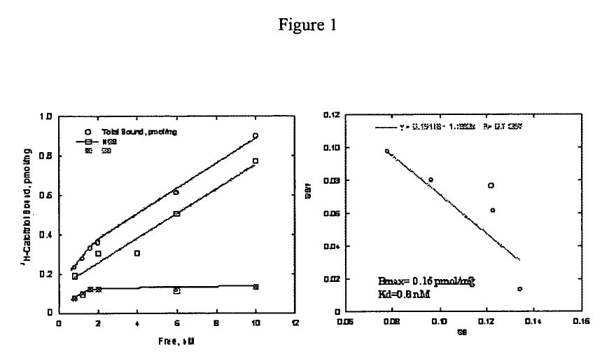

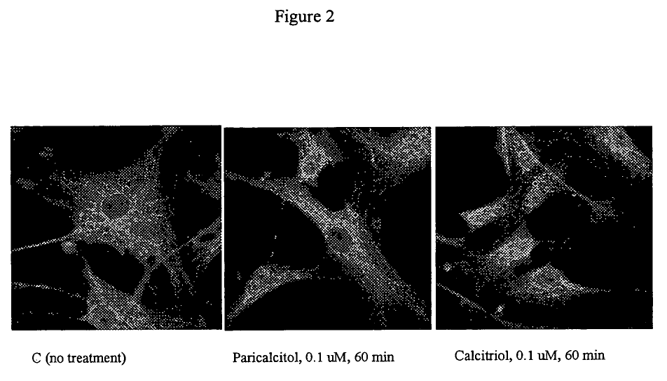

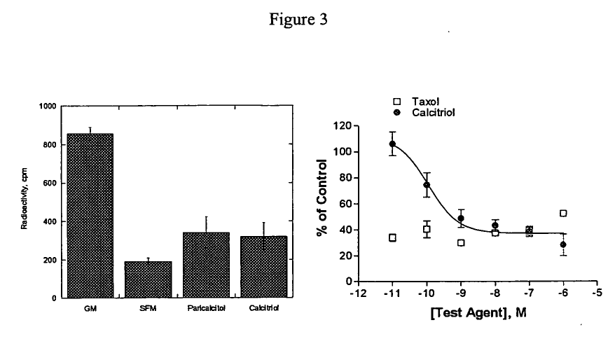

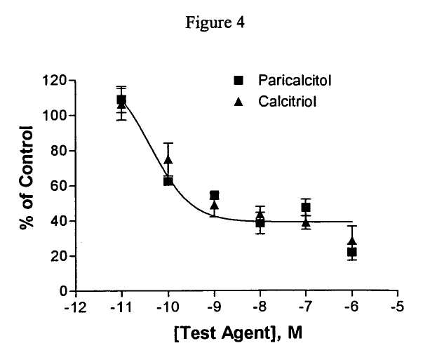

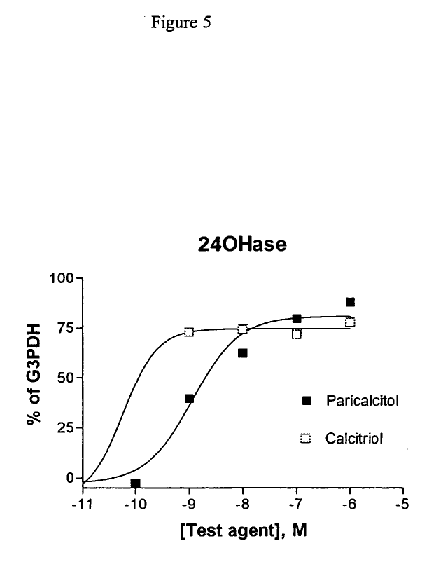

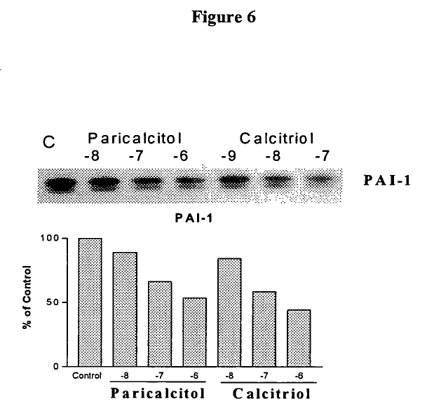

The vitamin D receptor was characterized in primary culture of CASMC and the functional effects of paricalcitol and calcitriol was studied on these cells. In VDR binding using cell extract prepared from these cells, the Bmax was 0. 16. μmol/mg, and the Kd 0.8 nM for [³H]-calcitriol (FIG. 1). Confocal microscopy using antibodies against VDR showed that VDR resided mainly in the cytoplasm in the absence of compounds that activate the VDR when cells were in the growth medium. Upon treatment by paricalcitol and calcitriol, VDR translocated into the nucleus, although not all cells responded to the treatment (See FIG. 2). Addition of a compound that activates a VDR resulted in inhibition of DNA synthesis (as determined by [³H]-thymidine incorporation), similar to the effect of serum free medium. The effect of paricalcitol and calcitriol on DNA synthesis was dose-dependent (See FIGS. 3 and 4). As a comparison, taxol (an anti-neoplastic agent) severely impacted DNA synthesis even at 0.01 nM (FIG. 3). As assessed by RT-PCR, calcitriol was ˜100-fold more potent in stimulating 24-OHase expression than paricalcitol (See FIG. 5). Interestingly, paricalcitol was slightly more potent in inhibiting DNA synthesis in these cells (EC₅₀=0.044±0.004 vs. 0.111±0.02 nM, FIG. 4). Also, paricalcitol was as potent as calcitriol in reducing the level of plasminogen activator inhibitor type-1 (PAI-1), one of the risk markers for coronary heart disease, which is enhanced in atherosclerotic plagues and co-localizes with macrophages (See FIG. 6). These results suggest that paricalcitol and calcitriol induce VDR translocation into the nucleus in CASMC, and paricalcitol is as potent as calcitriol in inhibiting cell proliferation and PAI-1 expression. Paricalcitol is usually dosed 3-4-fold higher than calcitriol in the clinical situation, which may translate into a more profound effect for paricalcitol in regulating the function of smooth muscle cells. Thus, the inhibition of smooth muscle cell proliferation would therefore be associated with a reduction in intimal hyperplasia in a mammal (as intimal hyperplasia is the result of a proliferation of smooth muscle cells.

### EXAMPLE 2

### Effect of Vitamin D Analogs on Vasculature

Materials and Methods

Cell cultures: Primary cultured human coronary artery smooth muscle cells (Cambrex) were grown to confluence in SMGM-2 containing 5.5 mM glucose, 5% FBS, 50 μg/ml gentamicin, 50 ng/ml amphotericin-B, 5 μg/ml insulin, 2 ng/ml human recombinant fibroblast growth factor, and 0.5 ng/ml human recombinant epidermal growth factor (growth medium) at 37° C. in a humidified 5% CO₂-95% air atmosphere. Cells were grown to >80% confluence and used within five passages.

Microarray: Total RNA was extracted from primary culture of human coronary artery smooth muscle cells grown in complete media and treated with or without 100 nM of paricalcitol or calcitriol for 30 hr (n=3 for each condition). While the yield of total RNA was low (˜2.0 gg total per sample), the RNAs were intact as judged by Agilent 2100 analysis. One μg of total RNA from each sample was used to prepare biotin-labeled cRNA target using standard Affymetrix protocols. Prepared cRNA targets were of good quality and quantity. The Affymetrix Human chip U133Av2 was used (22,000+ probe sets) and 10 μg cRNA target was applied to each array. After hybridization and chip scanning, the quality control data report (i.e., scaling factor, glyceraldehyde-3-phosphate dehydrogenase 5′/3′ ratio, noise, background) demonstrated that every array passed all quality criteria. Scanned images were loaded into the Rosetta Resolver 4.0 database and processed using the Resolver Affymetrix error model. Within Resolver the drug treated sample replicates (n=3) were informatically combined and ratios constructed relative to the combined control (no addition of drug) samples. The Resolver Affymetrix error model was used to develop each ratio and calculate a p-value. The Resolver error model is robust and includes reporter level, background and error values in the calculation of statistical significance. A combination of hierarchical clustering, gene ontology analysis and pathway mapping were used to assess the function of the regulated genes.

Primers and probes for Real-time Reverse Transcription-PCR: For quantitative PCR (qPCR), TaqMan™ probes that were 5′ labeled with the reporter 6-carboxyfluorescein (FAM) and 3′ labeled with the quencher tetramethylrhodamine (TAMRA) were used. The primer and probe sets were obtained from the Assay-on-Demand collection (Applied Biosytsems).

Real-time Reverse Transcription-PCR: PCR was performed with a 7900HT sequence detector (Applied Biosytsems). Each sample has a final volume of 25 μl containing 50 ng of cDNA, 0.4 mM each of the forward and reverse PCR primers and 0.1 mM of the TaqMan™ probe. Temperature conditions consisted of a step of 5 minutes at 95° C., followed by 40 cycles of 60° C. for 1 minute and 95° C. for 15 seconds. Data was collected during each extension phase of the PCR reaction and analyzed with the SDS software package (Applied Biosystems). Threshold cycles were determined for each gene. The expression level of target gene of interest was given as relative expression normalized against glyceraldehyde-3-phosphate dehydrogenase (GAPDH).

Thymidine incorporation: Cells were plated at 1×10⁶ cells/ml, 200 μl/well into 96-well plates (Costar). One day after plating, the cells were treated with test agents for 24 hr and then labeled with 0.3 μCi/well of ³H-thymidine for another 48 hr. Each well was washed with 0.3 ml/well of PBS, incubated with 0.2 ml/well of ice-cold 10% trichloroacetic acid (TCA) for 30 min at 4° C., and then followed by another wash of 0.2 ml/well of TCA. Samples were dissolved in MICROSCINT™ 20 (Packard) before counting.

1. Effects of Paricalcitol vs. Calcitriol:

Using a two-fold change in average difference in either the paricalcitol- or calcitriol-treated group as cutoff with p<0.05 for significantly modulated expression, a total of 176 target genes were identified. In the paricalcitol group, 115 and 61 genes were up- and down-regulated, respectively. In the calcitriol group, 116 and 60 genes were up- and down-regulated, respectively.

2. Functional clustering of modulated genes: Gene ontology analysis with EASE/DAVID (http://apps1.niaid.nih.gov/david/)

The paricalcitol data set was analyzed using the NIAID's Expression Analysis Systematic Explorer (EASE) program and the GenMAPP/MAPPFinder Pathway analysis program to assess the general effects of drugs on intracellular signaling and metabolic pathways. A number of functionally-related clusters was revealed. The results with EASE scores <0.05 (EASE score is upper bound of FisherExact score) show that cell proliferation and differentiation is one of the key clusters identified from this analysis.

3. Genes Present in the Cell Differentiation/Proliferation Gene Ontology Categories:

Disorders in smooth muscle cell proliferation play an important role in the restenosis/atherosclerosis process. Therefore, the genes indicated in the Gene Ontology categories of cell proliferation and differentiation are of special interest. Table 3 shows a cluster of genes involved in cell proliferation and differentiation. Potent modulation of multiple receptors involved in proliferation was evident.

TABLE 3
Genes present in the cell differentiation/proliferation Gene Ontology categories
Cell Differentiation

| | Paricalcitol | | Calcitriol | | | |
|---|---|---|---|---|---|---|
| Affy ID | FC/p-value | | FC/p-value | | GENENAME | GENE ONTOLOGY CLASSIFICATIONS (selected) |
| 205789_at | 5.38 | 0.003 | 6.95 | 0.001 | CD1D antigen, d polypeptide | T-cell selection; antigen presentation, endogenous lipid antigen; |
| | | | | | | beta-2-microglobulin binding; positive regulation of innate immune |
| | | | | | | response; receptor activity |
| 220935_s_at | 2.91 | 0.001 | 2.67 | 0.000 | CDK5 regulatory subunit | brain development; cytoskeleton; neuronal Cdc2-like kinase binding; |
| | | | | | associated protein 2 | regulation of neuron differentiation |
| 220266_s_at | 2.37 | 0.000 | 2.67 | 0.000 | Kruppel-like factor 4 (gut) | mesoderm cell fate determination; negative regulation of cell |
| | | | | | | proliferation; negative regulation of transcription, transcriptional |
| | | | | | | activator activity; transcriptional repressor activity; zinc ion binding |
| 206404_at | 1.88 | 0.000 | 2.07 | 0.000 | fibroblast growth factor 9 (glia- | cell differentiation; cell proliferation; cell-cell signaling; growth |
| | | | | | factor activating factor) | activity; heparin binding; regulation of cell cycle; |
| | | | | | signal transduction | |
| 221530_s_at | −1.80 | 0.000 | −2.00 | 0.000 | basic helix-loop-helix domain | cell differentiation; cell proliferation; organogenesis; regulation of |
| | | | | | containing, class B, 3 | transcription, DNA-dependent; transcription factor activity |
| 207030_s_at | −2.18 | 0.000 | −2.13 | 0.000 | cysteine and glycine-rich protein 2 | cell differentiation; cell growth; cell proliferation; muscle |
| | | | | | | development; nucleus |
| 211126_s_at | −2.33 | 0.000 | −2.21 | 0.000 | cysteine and glycine-rich protein 2 | cell differentiation; cell growth; cell proliferation; muscle |
| | | | | | | development; nucleus |
| 203373_at | −2.39 | 0.000 | −2.50 | 0.000 | suppressor of cytokine signaling 2 | growth hormone receptor binding; insulin-like growth factor receptor |
| | | | | | | binding; regulation of cell growth; regulation of signal transduction |
| 203372_s_at | −2.70 | 0.000 | −2.29 | 0.000 | suppressor of cytokine signaling 2 | growth hormone receptor binding; insulin-like growth factor receptor |
| | | | | | | binding; regulation of cell growth; regulation of signal transduction |
| 209540_at | 4.89 | 0.000 | 4.39 | 0.000 | insulin-like growth factor 1 | DNA replication; RAS protein signal transduction; cell motility; |
| | | | | | (somatomedin C) | glycolate metabolism; growth factor activity; muscle development;; |
| | | | | | | positive regulation of cell proliferation |
| 216953_s_at | 4.64 | 0.000 | 6.63 | 0.000 | Wilms tumor 1 | negative regulation of cell cycle; regulation of transcription; |
| | | | | | | transcription factor activity |
| 213524_s_at | 3.46 | 0.000 | 3.69 | 0.000 | putative lymphocyte G0/G1 switch | molecular_function unknown; regulation of cell cycle |
| | | | | | gene | |
| 211577_s_at | 3.43 | 0.000 | 3.47 | 0.000 | insulin-like growth factor 1 | DNA replication; RAS protein signal transduction; cell motility; |
| | | | | | (somatomedin C) | glycolate metabolism; growth factor activity; muscle development;; |
| | | | | | | positive regulation of cell proliferation |
| 206067_s_at | 3.32 | 0.000 | 3.55 | 0.000 | Wilms tumor 1 | negative regulation of cell cycle; regulation of transcription; |
| | | | | | | transcription factor activity |
| 210059_s_at | 3.26 | 0.000 | 3.27 | 0.000 | mitogen-activated protein kinase | MAP kinase activity; antimicrobial humoral response; cell cycle; |
| | | | | | 13 | protein serine/threonine kinase activity; regulation of translation; |
| | | | | | | response to stress; signal transduction |
| 209542_x_at | 3.24 | 0.000 | 3.23 | 0.000 | insulin-like growth factor 1 | DNA replication; RAS protein signal transduction; cell motility; |
| | | | | | (somatomedin C) | glycolate metabolism; growth factor activity; muscle development;; |
| | | | | | | positive regulation of cell proliferation |
| 209747_at | 2.80 | 0.000 | 2.83 | 0.000 | transforming growth factor, beta 3 | cell growth; cell proliferation; cell-cell signaling; organogenesis; |
| | | | | | | regulation of cell cycle; signal transduction |
| 206954_at | 2.72 | 0.003 | 2.96 | 0.001 | Wilms tumor associated protein | cell proliferation |
| 209541_at | 2.63 | 0.000 | 2.78 | 0.000 | insulin-like growth factor 1 | DNA replication; RAS protein signal transduction; cell motility; |
| | | | | | (somatomedin C) | glycolate metabolism; growth factor activity; muscle development;; |
| | | | | | | positive regulation of cell proliferation |
| 209909_s_at | 2.48 | 0.000 | 2.91 | 0.000 | transforming growth factor, beta 2 | cell growth; cell proliferation; cell-cell signaling; regulation of cell |
| | | | | | | cycle; signal transduction |
| 210058_at | 2.40 | 0.000 | 2.27 | 0.000 | mitogen-activated protein kinase | MAP kinase activity; antimicrobial humoral response; cell cycle; |
| | | | | | 13 | protein serine/threonine kinase activity; regulation of translation; |
| | | | | | | response to stress; signal transduction |
| 220266_s_at | 2.37 | 0.000 | 2.67 | 0.000 | Kruppel-like factor 4 (gut) | mesoderm cell fate determination; negative regulation of cell |
| | | | | | | proliferation; negative regulation of transcription, transcriptional |
| | | | | | | activator activity; transcriptional repressor activity; zinc ion binding |
| 209805_at | 2.18 | 0.024 | 2.85 | 0.001 | PMS2 postmeiotic segregation | ATP binding; DNA binding; mismatch repair; negative regulation of |
| | | | | | increased 2 (S. cerevisiae) | cell cycle |
| 220407_s_at | 1.88 | 0.060 | 2.97 | 0.000 | transforming growth factor, beta 2 | cell growth; cell proliferation; cell-cell signaling; regulation of cell |
| | | | | | | cycle; signal transduction |
| 206404_at | 1.88 | 0.000 | 2.07 | 0.000 | fibroblast growth factor 9 (glia- | cell differentiation; cell proliferation; cell-cell signaling; heparin |
| | | | | | activating factor) | binding; regulation of cell cycle; signal transduction |
| 212332_at | −1.37 | 0.202 | 1.10 | 0.681 | retinoblastoma-like 2 (p130) | DNA binding; cell cycle; negative regulation of cell cycle; nucleus; |
| | | | | | | protein binding; regulation of transcription |
| 209114_at | −1.79 | 0.125 | −4.34 | 0.003 | tetraspan 1 | cell adhesion; cell motility; cell proliferation; integral to membrane |
| 221530_s_at | −1.80 | 0.000 | −2.00 | 0.000 | basic helix-loop-helix domain | cell differentiation; cell proliferation; organogenesis; regulation of |
| | | | | | containing, class B, 3 | transcription, DNA-dependent; transcription factor activity |
| 213067_at | −1.80 | 0.000 | −2.03 | 0.000 | myosin heavy polypeptide 10, non- | ATP binding; actin binding; calmodulin binding; cellular |
| | | | | | muscle | morphogenesis; cytokinesis; motor activity; myosin |
| 207030_s_at | −2.18 | 0.000 | −2.13 | 0.000 | cysteine and glycine-rich protein 2 | cell differentiation; cell growth; cell proliferation; muscle |
| | | | | | | development; nucleus |
| 211126_s_at | −2.33 | 0.000 | −2.21 | 0.000 | cysteine and glycine-rich protein 2 | cell differentiation; cell growth; cell proliferation; muscle |
| | | | | | | development; nucleus |
| 204774_at | −2.49 | 0.000 | −1.77 | 0.001 | ecotropic viral integration site 2A | cell proliferation; integral to membrane; transmembrane receptor |
| | | | | | | activity |
| 204827_s_at | −2.75 | 0.010 | −1.09 | 0.781 | cyclin F | cytokinesis; mitosis; nucleus; regulation of cell cycle |
| 210381_s_at | −3.04 | 0.077 | −1.65 | 0.340 | cholecystokinin B receptor | cytosolic calcium ion concentration elevation; feeding behavior; |
| | | | | | | gastrin receptor activity; phosphatidylinositol 3-kinase regulator |
| | | | | | | activity; phospholipase C activation; positive regulation of cell |
| | | | | | | proliferation; rhodopsin-like receptor activity; sensory perception |

Previous studies have demonstrated that the Wilms tumor 1 (WT1) gene is a positive regulator of VDR expression as well as a negative regulator of insulin-like growth factor 1 (IGF 1) receptor expression and function. Additionally, there is evidence that IGF1 can negatively regulate the expression of WT1 at the transcriptional level. It was also noted that transforming growth factor beta (TGFβ) was up-regulated, which would be expected to have a negative regulatory effect on proliferation. Furthermore, negative regulators of proliferation such as KLF4 were modestly up-regulated. These results suggest that vitamin D analogs are involved in modulating the proliferation of smooth muscle cells.

4. Selected Genes Linked to Cardiovascular Functions:

Selected genes regulated by paricalcitol and calcitriol that have been previously shown to play roles in cardiovascular functions are shown in Table 4 below.

TABLE 4

| | | Paricalcitol | | Calcitriol | |
|---|---|---|---|---|---|
| Sequence description | GenBank ID | FC/p-value | | FC/p-value | |
| fibroblast growth factor receptor 3 (achondroplasia, | NM_000142 | 3.4 | 0.000 | 1.9 | 0.137 |
| thanatophoric dwarfism) | | | | | |
| thrombomodulin | NM_000361 | 3.0 | 0.000 | 3.3 | 0.000 |
| transforming growth factor, beta 3 | J03241 | 2.9 | 0.000 | 2.9 | 0.000 |
| insulin-like growth factor 1 (somatomedin C) | NM_000618 | 2.5 | 0.000 | 2.8 | 0.000 |
| transforming growth factor, beta 2 | NM_003238 | 2.5 | 0.000 | 2.9 | 0.000 |
| endothelin receptor type B | NM_000115 | 2.3 | 0.000 | 3.4 | 0.000 |
| prostaglandin-endoperoxide synthase 1 (prostaglandin G/H | NM_000962 | 2.0 | 0.000 | 2.2 | 0.000 |
| synthase and cyclooxygenase) | | | | | |
| fibroblast growth factor 9 (glia-activating factor) | NM_002010 | 1.9 | 0.000 | 2.1 | 0.000 |
| angiopoietin 1 | NM_001146 | 1.7 | 0.000 | 2.0 | 0.000 |
| matrix metalloproteinase 14 (membrane-inserted) | NM_004995 | 1.1 | 0.694 | 2.1 | 0.009 |
| coagulation factor II (thrombin) receptor-like 1 | NM_005242 | −2.1 | 0.001 | −1.7 | 0.014 |
| phospholamban | NM_002667 | −2.4 | 0.000 | −2.6 | 0.000 |
| natriuretic peptide precursor B | NM_002521 | −2.5 | 0.000 | −2.0 | 0.000 |
| oxytocin receptor | NM_000916 | −2.6 | 0.000 | −3.0 | 0.000 |
| thrombospondin 1 | AI812030 | −2.7 | 0.000 | −3.2 | 0.000 |

Thrombomodulin (TM) expression was increased by both drugs. The mRNA expression of thrombospondin 1 (THBS 1, an extracellular matrix protein), MMP 14 (one of the matrix metalloproteinases) and thrombin receptor-like 1 was down-regulated. Prostaglandin-endoperoxide synthase 1 (COX1) and type-B endothelin receptor were increased. As a comparison, natriuretic peptide prescurson B (BNP) was down-regulated. The natriuretic peptides are known to play a role in the regulation of cardiovascular, renal and endocrine homeostasis. Furthermore, several genes such as angiopoietin 1, IGF, and TGFβ that have been implicated to play roles in endothelial regeneration were altered by paricalcitol and calcitriol.

5. Confirmation of Gene Microarray Results by Real-time RT-PCR:

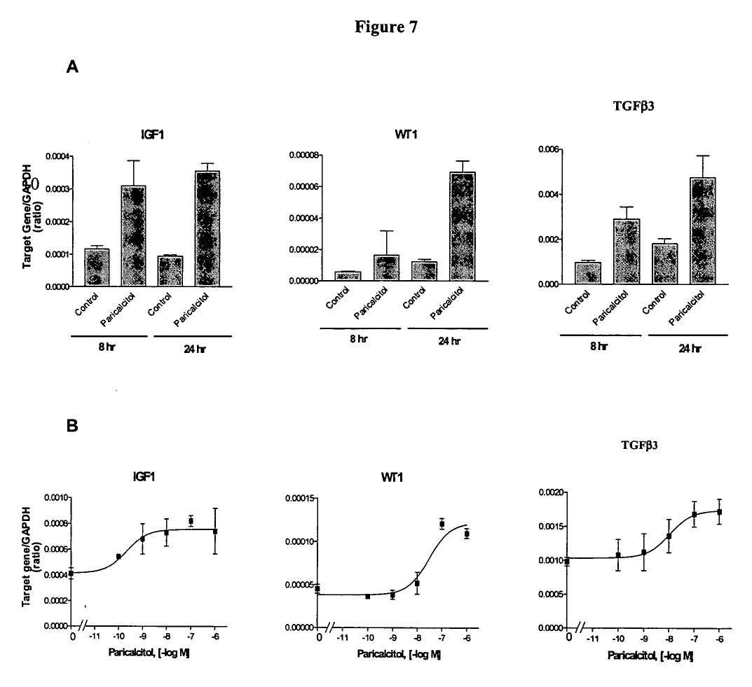

As a validation of the results, real-time RT-PCR was employed to examine the effect of paricalcitol on 3 genes, IGF1, WT1, TGFβ3, that are known to be involved in modulation of cell proliferation. As shown in FIG. 7, paricalcitol up-regulated the expression of these 3 genes in a time- and dose-dependent manner. The EC₅₀ values of paricalcitol on IGF1, WT1, and TGFβ3 were 0.2, 29.7 and 10.6 nM, respectively.

6. Effects of Paricalcitol and Calcitriol on Cell Proliferation:

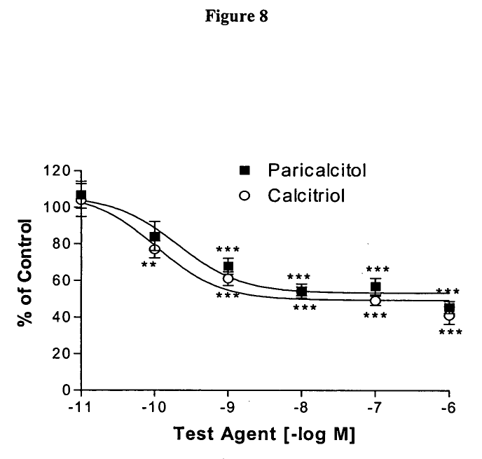

In order to further understand the significance of the above observation that vitamin D analogs likely regulate smooth muscle cell proliferation, an examination was made of the effects of paricalcitol and calcitriol on ³H-thymidine incorporation. FIG. 8 shows that both paricalcitol and calcitriol inhibited thymidine incorporation in a dose-dependent manner. The EC₅₀ were determined to be 0.2 and 0.11 nM for paricalcitol and calcitriol, respectively. At 10 nM, both paricalcitol and calcitriol inhibited thymidine incorporation by 46% (when compared to cells in growth medium).

The above results show that thrombomodulin (TM) is up-regulated in human coronary artery SMC. Previously, it has been shown that an active form of vitamin D up-regulates TM gene expression and down-regulates tissue factor gene expression in monocytic cells. Recently, it was shown that, in the VDR KO mice, the TM gene in the aorta, liver, and kidney was down-regulated, while tissue factor mRNA expression in the liver and kidney was up-regulated independent of plasma calcium levels. The above results also show that the extracellular matrix protein thrombospondin 1, MMP 14 and thrombin receptor-like 1 are down-regulated. Decreased fibrinolytic capacity and increased thrombogenicity have been shown to accelerate atherogenesis. Taken together, the present results demonstrate that vitamin D analogs may suppress thrombogenicity and enhance fibrinolysis, suggesting that these drugs are beneficial in regulating intimal plaque formation during the restenosis/atherosclerosis process.

Further, it has been found that several genes involved in endothelial regeneration and vessel relaxation are regulated by vitamin D analogs. The type-B endothelin receptor, which plays a role in enhancing NO release and vessel relaxation, is up-regulated by paricalcitol and calcitriol. Oxytocin receptor, which is the receptor for oxytocin and plays a role in contraction, is down-regulated. Cyclooxygenase is known to catalyze the initial step in converting arachidonic acid to prostaglandins and thromboxane, which impacts vessel constriction/relaxation differently. Our result demonstrates that prostaglandin-endoperoxide synthase 1 (prostaglandin G/H synthase and cyclooxygenase) is up-regulated by both paricalcitol and calcitriol. Furthermore, several genes such as angiopoietin 1, insulin-like growth factor, and transforming growth factor are altered. These results suggest that VDR activation by vitamin D analogs plays a potential role in vessel relaxation and endothelial regeneration.

It is known that aberrant growth of SMC plays an important role in diseases such as atherosclerosis and restenosis. In this study, findings that gene clusters related to proliferation and differentiation are significantly modulated by paricalcitol and calcitriol suggest that vitamin D analogs will likely regulate the growth of smooth muscle cells. Indeed, the above results show that both calcitriol and paricalcitol dose-dependently inhibit the proliferation of human coronary artery smooth muscle cells cultured in growth medium.

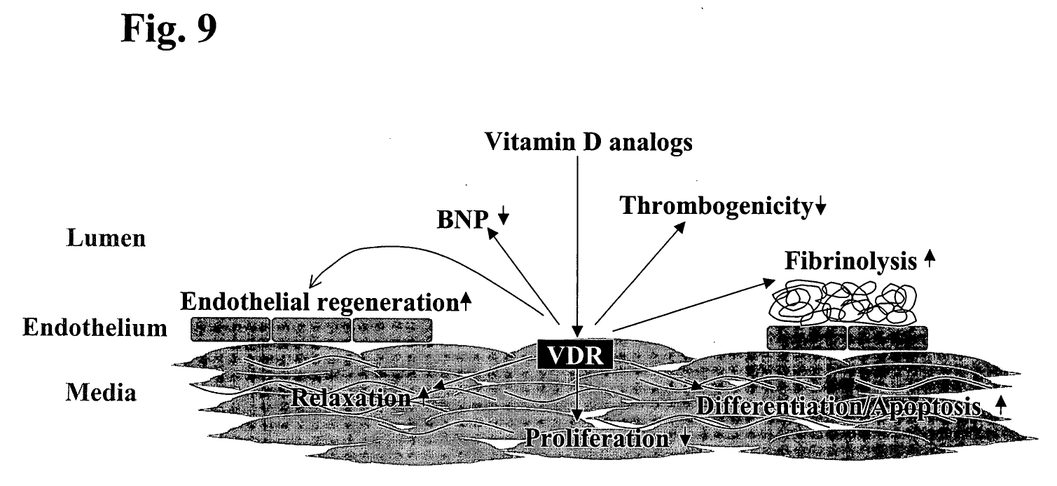

Results from this study suggest that vitamin D analogs exert beneficial effects on the vasculature via regulation of smooth muscle cell proliferation/differentiation, thrombosis, fibrinolysis, vessel relaxation and endothelial regeneration (FIG. 9).

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. A method of reducing smooth muscle cell proliferation in a mammal suffering from a vascular disease, wherein said vascular disease causes a narrowing or obstruction of a blood vessel, the method comprising the step of administering to said mammal a therapeutically effective amount of a compound that activates a vitamin D receptor (VDR).

2. The method of claim 1 wherein the compound is administered orally or intravenously.

3. The method of claim 1 wherein the compound is administered as a coating on a stent.

4. The method of claim 1 wherein the compound is administered as a coating on a graft or implant.

5. The method of claim 1 wherein the compound is a vitamin D compound.

6. The method of claim 5 wherein the vitamin D compound is administered in amount of from about 0.04 to about 0.24 mcg/kg, intravenously, 3 times per week.

7. The method of claim 5 wherein the vitamin D compound is a vitamin D metabolite or analog.

8. The method of claim 7 wherein the vitamin D metabolite or analog is 1,25-dihydroxy vitamin D₃ or 19-nor-1α, 25-dihydroxyvitamin D₂.

9. A method of reducing intimal hyperplasia in a mammal suffering from chronic kidney disease, the method comprising the step of administering to said mammal a therapeutically effective amount of a compound that activates a vitamin D receptor (VDR).

10. The method of claim 9 wherein the compound is administered orally or intravenously.

11. The method of claim 9 wherein the compound is administered as a coating on a stent.

12. The method of claim 9 wherein the compound is administered as a coating on a vascular access graft.

13. The method of claim 9 wherein the compound is a vitamin D compound.

14. The method of claim 13 wherein the vitamin D compound is administered in amount of from about 0.04 to about 0.24 mcg/kg, intravenously, 3 times per week.

15. The method of claim 13 wherein the vitamin D compound is a vitamin D metabolite or analog.

16. The method of claim 15 wherein the vitamin D metabolite or analog is 1,25-dihydroxy vitamin D₃ or 19-nor-1α, 25-dihydroxyvitamin D₂.

17. A method of reducing restenosis in a mammal that has undergone angioplasty, the method comprising the step of administering to said mammal a therapeutically effective amount of a compound that activates a vitamin D receptor (VDR).

18. The method of claim 17 wherein the compound is administered orally or intravenously.

19. The method of claim 17 wherein the compound is administered as a coating on a stent.

20. The method of claim 17 wherein the compound is administered as a coating on a graft or implant.

21. The method of claim 17 wherein the compound is a vitamin D compound.

22. The method of claim 21 wherein the vitamin D compound is administered in amount of from about 0.04 to about 0.24 mcg/kg, intravenously, 3 times per week.

23. The method of claim 21 wherein the vitamin D compound is a vitamin D metabolite or analog.

24. The method of claim 23 wherein the vitamin D metabolite or analog is 1,25-dihydroxy vitamin D₃ or 19-nor-1α, 25-dihydroxyvitamin D₂.

25. A method of reducing intimal hyperplasia in a mammal that has undergone coronary artery bypass surgery, the method comprising the step of administering to said mammal a therapeutically effective amount of a compound that activates a vitamin D receptor (VDR):

26. The method of claim 25 wherein the compound is administered orally or intravenously.

27. The method of claim 25 wherein the compound is administered as a coating on a stent.

28. The method of claim 25 wherein the compound is administered as a coating on a graft or implant.

29. The method of claim 25 wherein the compound is a vitamin D compound.

30. The method of claim 29 wherein the vitamin D compound is administered in amount of from about 0.04 to about 0.24 mcg/kg, intravenously, 3 times per week.

31. The method of claim 29 wherein the vitamin D compound is a vitamin D metabolite or analog.

32. The method of claim 31 wherein the vitamin D metabolite or analog is 1,25-dihydroxy vitamin D₃ or 19-nor-1α, 25-dihydroxyvitamin D₂.

33. A method of reducing intimal hyperplasia in a mammal suffering from chronic kidney disease and who has a need for hemodialysis that requires implementation of a vascular access device, the method comprising the step of administering to said mammal a therapeutically effective amount of a compound that activates a vitamin D receptor (VDR).

34. The method of claim 33 wherein the compound is administered orally or intravenously.

35. The method of claim 33 wherein the compound is administered as a coating on a vascular access device.

36. The method of claim 33 wherein the compound is a vitamin D compound.

37. The method of claim 36 wherein the vitamin D compound is administered in amount of from about 0.04 to about 0.24 mcg/kg, intravenously, 3 times per week.

38. The method of claim 36 wherein the vitamin D compound is a vitamin D metabolite or analog.

39. The method of claim 38 wherein the vitamin D metabolite or analog is 1,25-dihydroxy vitamin D₃ or 19-nor-1α, 25-dihydroxyvitamin D₂.
